(19) United States
(12) Patent Application Publication
(10) Pub. No.: US 2025/0213469 A1
(43) Pub. Date: Jul. 3, 2025

(54) DOSAGE FORMS OF ACTIVE SUBSTANCES USED AGAINST VIRAL DISEASES IN DRY POWDER INHALER DEVICE FOR SYMPTOMS CAUSED BY COVID-19 AND OTHER VIRAL LUNG DISEASES

(71) Applicant: PULMOCURES ILAC EGITIM DANISMANLIK A.S., Avcilar/Istanbul (TR)

(72) Inventor: Ayca Yildiz Pekoz, Besiktas/Istanbul (TR)

(21) Appl. No.: 18/848,271

(22) PCT Filed: Mar. 18, 2022
(86) PCT No.: PCT/TR2022/050249

§ 371 Date: Sep. 18, 2024

(51) Int. Cl. A61K 9/00; A61K 47/12; A61K 47/26; A61K 47/32

(52) U.S. Cl. CPC A61K 9/0075; A61K 47/12; A61K 47/26; A61K 47/32

(57) ABSTRACT

The present invention relates to a pharmaceutical composition in inhalable pre-metered dry powder form that comprises at least one excipient (for example carrier) and at least one active substance used against viral diseases to locally administer it to the lungs for use in the treatment of symptoms of viral lung diseases including COVID-19 caused by Severe Acute Respiratory Syndrome-Coronavirus-2 (SARS-COV-2) by means of dry powder inhaler through inhalation. The present invention also relates to methods for preparation of said pharmaceutical composition and the use of these active substances in symptoms caused by COVID-19. similar viral lung diseases, influenza.

### TECHNICAL FIELD OF THE INVENTION

The present invention relates to a pharmaceutical composition comprising at least one excipient and at least one active substance used against viral diseases in inhalable pre-metered dry powder form, and direct delivery of this pharmaceutical composition to the lungs with dry powder inhaler (DPI) in the treatment of symptoms caused by COVID-19 and other viral lung diseases. The present invention also relates to methods for preparation of said pharmaceutical composition and the use of these active substances in symptoms caused by COVID-19, similar viral lung diseases.

### STATE OF THE ART

COVID-19 viral infection caused by a new type of coronavirus called SARS-COV-2, identified as a new member of the coronavirus family, has been reported as the fastest spreading global epidemic factor the world has witnessed in the past century. SARS-CoV-2 is an infectious and extremely pathogenic coronavirus that causes pneumonia in humans, an epidemic of severe respiratory tract infection. The viral respiratory disease caused by the SARS-COV-2 virus and the most common symptoms thereof, which manifest themselves as high fever, cough, and respiratory distress (dyspnea, difficulty in breathing), has been defined as COVID-19.

COVID-19 disease caused by SARS-COV-2 can affect many organs such as the lungs, brain, kidney and liver. The organ with the most mortality effect is the lung. The virus directly attacks the lungs and initiates lung damage within 5 days. In severe cases, patients usually die due to respiratory failure. Pathogens such as viruses can reach and settle in the lungs through the respiratory tract and cause serious infections in this region. In the treatment of these microbial and viral-based diseases that show high involvement in the lung and cause severe lung infections, application options with a formulation and systemic effect are generally used in conventional dosage form (tablets, parenteral drugs etc.). The main reason for this is that the active substances used in the treatment of diseases caused by these infectious agents are designed or produced in the form of conventional dosage form along with a systemic effect.

The main disadvantages of the conventional dosage forms that are commercially available for use in the relevant symptoms in the treatment of acute lung diseases (e.g. COVID-19, pneumonia) or chronic diseases (COPD, asthma) can be summarized as follows;
1) Insufficient accumulation of active substance in lung tissue; it has been proven that drugs administered in both tablet dosage form and parenteral form have low accumulation in the lung bronchoalveolar lavage fluid and lung tissue.
2) The possibility of the active substances used to cause toxic effects in the whole body due to systemic administration.
3) Due to the inability to swallow tablet forms in intubated patients and the difficulty in swallowing the tablet form in pediatric patients, the actual practice in hospitals based on medical treatment guidelines is the method of crushing these forms. However, even if it is practical, this method reduces bioavailability by up to 50 percent. Our present invention provides effective treatment opportunity to these patients by eliminating the bioavailability reductions caused by this method.
4) Low compliance with conventional dosage forms in pediatric patients due to the bad taste of active ingredients and difficulty swallowing,
5) Due to the fact that the tablet forms are mostly film-coated, the correct dosage cannot be adjusted when applied by splitting or crushing.
6) Parenteral application could only be obtained by special health care providers, and could not be done by the patient himself.

Clinicians and researchers have turned to alternative local application options to conventional dosage forms in the applications of lung diseases due to the above-mentioned disadvantages. Currently, administration of drugs in the deep lung region is obtained through inhalation devices such as:
nebulizers, in which the drug is dissolved or dispersed in the form of suspension and carried to the lung as nebulized droplets,
powder inhalers, capable of delivering the drug present in the inhaler as dry micronized particle,
pressurized inhalers, through which the drug—again in the form of droplets of solution or suspension is carried to the deep lung region by an inert gas expanded rapidly in air by a pressurized canister.

It is known that COVID-19 primarily affects the lungs. In severe cases, patients usually die due to respiratory failure. Therefore, the effective drug dose must be delivered to the lungs for treatment. However, existing oral use of antiviral substances that is used against COVID-19, for example favipiravir, do not directly target the lungs and causes systemic effects. This situation reduces the effectiveness of the treatment and increases the side effects during the treatment. Bocan et al., in a biodistribution study of [¹⁸F] Favipiravir, showed that the active substance was retained lowest in the lungs and the most in the kidney, liver and stomach¹.

The choice of a drug that is used in the treatment of lung diseases (as in any organ or tissue) is primarily for the local treatment of said organ or tissue. Local treatment ensures the drugs to be used are effective only in the determined organ or tissue, and other parts of the body are not exposed to the drug systemically. The administration results in more effective although the active substances are applied in lower amounts and the side effects thereof are reduced by means of the local administration of the drug. Clinicians and researchers have turned to options of local application as an alternative to conventional dosage forms in the applications of lung diseases due to the disadvantages mentioned above.

In the prior art, dry powder inhalers (DPIs) are used in treatment of numerous diseases. There are two main kinds of DPIs (I) monodose DPIs in which the doses of active ingredient (mixed or not with an excipient) are preseparated by filling in individual gelatine capsules and (ii) multidose DPIs in which the drug (mixed or not with an excipient) is filled into a reservoir, the amount of drug delivered per actuation being controlled by a dosing chamber. The active ingredient is either pelletized or mixed with a coarse excipient.

In the prior art, CN111297838A patent application relates to an antiviral drug inhalation spray, and in particular to an anti-coronavirus COVID-19 drug inhalation spray. The inhalation spray comprises the following components in percentage by mass of 0%-30% of antivirus activity agents, 0%- 30% of auxiliary agents, 0%- 30% of taste masking agents and the balance solvents, wherein the content of the antivirus activity agents and the content of the auxiliary agents are not 0% at the same time. With this inhalation spray, during outbreak period of epidemic corona viruses and other viruses, people do not need to occupy medical resources in short supply and only need to inhale the antivirus medicines into respiratory tracts. However, CN111297838A uses “inhalation spray” in a general term and do not indicate any particular formulation properties such as suspension, emulsion, foam etc. besides it is a general spray formulation and do not provide spray forms with increased effectiveness.

In the prior art, CN104288154B patent relates to a pharmaceutical composition of favipiravir containing different particle size ranges. The invention solves the problem of dissolution of favipiravir preparations in water through a special particle size distribution and preparation process. CN104288154B discloses a tablet or capsule composition using different portions of excipients with varying size distribution. Since it explains an oral delivery method, the composition will undergo gastrointestinal degradation and the lung will receive the least amount of drug. This oral medication form causes problems of using high doses of antivirals and therefore numerous side effects of these antivirals.

In EP1311243A2 patent application, a process for the production of powdered mannitol with improved flow characteristics for use in inhalation systems, and mannitol having a particle shape specific for this process is disclosed. The dry powder inhaler formulations employing the mannitol can be used in gene therapy, for treating pain including headache and migraines, for treating Alzheimer's, cancer, or with cytostatics, antiallergics, antidiabetics, antibiotics, bronchodilators, antitussives, antiasthmatics, steroids, sedatives, physiologically active peptides or proteins, growth hormones, antiinfectives or antivirals. In this invention, use of mannitol as a carrier in DPI's and other inhaler routes are explained. However, specific formulations and active substances against viral diseases are not explained in this application.

There are many active substances used against viral diseases that are also used in COVID-19 outbreak. In the prior art there are many studies that explain details of the use of active substances used against viral diseases such as favipiravir, umifenovir, molnupiravir, ritonavir, hydroxychloroquine etc. in COVID-19, their dosage, contraindications and interactions with other medications. However, they are generally focusing on oral drug delivery routes in the form of an oral tablet. As a result of this route, a high dose treatment guide is necessary and according to present studies, it is found that oral delivery route is an insufficient treatment route for favipiravir and other active substances used against viral lung diseases.

The prior art contains problems such as insufficient treatment in viral lung diseases, especially in COVID-19 with active substances used against viral diseases such as favipiravir, umifenovir, molnupiravir etc. by oral route, the necessity of using high doses of these antivirals in oral tablets and therefore numerous side effects of these antivirals and low efficacy of them in medicaments. Due to these problems in prior art, developments in the usage, delivery and administration of these active substances for the effective treatment of viral lung diseases, especially for COVID-19 is needed in this technical field.

### Brief Description and Objects of the Invention

The present invention relates to a pharmaceutical composition comprising at least one active substance in inhalable pre-metered dry powder form and at least one excipient, preferably loaded in a moisture-tight, dry container, and direct delivery of this pharmaceutical composition to the lungs with dry powder inhaler (DPI) in the treatment of symptoms caused by COVID-19 and other viral lung diseases. The active substance can be used alone or in combination with and/or mannitol.

The most important object of the present invention is to provide effective treatment of symptoms caused by COVID-19 and other viral lung diseases. The present invention allows administration of the active substance used against viral diseases locally and directly to the lung in the treatment of symptoms by using DPI. Since the present invention provides a targeted delivery of active substance with inhalation in pre-metered dry powder form, it has many advantages compared to other administration routes (oral, parenteral, etc.), thereby, it provides more effective treatment. In the present invention, since the active substance used against viral diseases will be targeted directly to the lungs in a form of inhalable pre-metered dry powder, more effective treatment is provided compared to prior art.

One of the main advantages of DPI is that they are breath-actuated delivery systems, they do not require co-ordination of actuation. Since release of the drug is dependent on the patient own inhalation; DPIs do not contain propelants acting as environmental hazards, and the velocity of the delivered particles is the same or lower than that of the flow of inspired air, so making them more prone to follow the air flow than the faster moving MDI particles, there by reducing upper respiratory tract deposition.

Another object of the present invention is to maximize the bioavailability of the active sustance and to ensure that the active substance used against viral diseases are used effectively in lower doses with higher efficacy and lower side effects. Bioavailability is maximized by direct administration to the target area, while minimizing the side effects and also minimizing the cost of the drug by local administration of smaller doses via the pulmonary route, compared to the oral and parenteral route. In the present invention, the drug efficacy increases, and side effects of the drug, which may occur systemically are reduced by means of its local administration compared to the oral and parenteral routes. The present invention overcomes the insufficient oral drug delivery route of favipiravir and/or other active substances, further decreases the required dosage treatment thus reducing unwanted side effects. Improved patient compliance is expected with the present invention since the required dosage amount is decreased greatly when compared to oral route of administration.

Another object of the present invention is to have a fast onset of action for the treatment of viral lung diseases. In the present invention, delivery of the active substance used against viral diseases through pulmonary route increases the bioavailability since the effect of liver first pass is eliminated. This is because the pulmonary route is an optimal route of administration for drugs that are poorly absorbed or quickly metabolized through the oral route.

Another object of the present invention is to provide an applicable method for patients who have difficulty swallowing tablets. The present invention provides an inhaled route of delivery for active substances, therefore the patients having difficulty in swallowing tablets can easily get the treatment by inhalation. While an oral tablet undergoes gastrointestinal system degradation, an inhaled form directlys targets the lungs.

In addition, another advantage of the present invention is the ease of dosing and logistics to be provided which brings great speed and efficiency to the health system under pandemic conditions. Since dosing of antiviral active substances to be used in the treatment is made possible by the patients in their home, the treatment efficiency will increase and the accumulation in the hospital will be reduced by shortening the recovery times.

In compared to prior art, the present invention uses sugar alcohol components, to provide mucolytic action etc. except taste masking. However, the formulations of prior art generally use sugar alcohol components as a taste masking agent. The present invention provides active pharmaceutical ingredient (API) and its dry powder form which ensures delivering the drug present in the inhaler as dry micronized particles to the deeper part of the lungs; whereby patent application mentioned in the prior art only claims that spray is useful for preventing viral infection and transmission of coronavirus COVID-19. In addition, the present invention targets inside the lung region by using a dry powder inhaler. In this case, the drug used against viral diseases provides local effects in the lung area. Also, since less dosage will be used in inhaled method of the present invention, side effects seen with the active ingredient will decrease as well.

Since the composition subject to the invention is in a dry powder dosage form, the problem of stability in the invention is much less common than nebulizers, soft mist inhalers and pMDIs. In addition, the propellant in the nebulizers of the prior art creates a coldness effect in patients. This undesirable coldness effect does not occur in the dry powder inhaler.

The present invention overcomes the problems present in the prior art by making developments in the usage, delivery and administration of active substances used against viral diseases for the effective treatment of viral lung diseases, especially COVID-19. The present invention provides an effective treatment of symptoms caused by viral lung diseases, especially COVID-19 with inhalation route, a fast onset of action compared to prior art, an effective use in lower doses with higher efficacy, lower side effects of active substances used against viral diseases and easy application method for patients who have difficulty swallowing tablets.

### DETAILED DESCRIPTION OF THE INVENTION

The present invention relates to a pharmaceutical composition in inhalable pre-metered dry powder form that comprises at least one excipient (for example carrier) and at least one active substance used against viral diseases to locally administer it to the lungs for use in the treatment of symptoms of viral lung diseases including COVID-19 caused by Severe Acute Respiratory Syndrome-Coronavirus-2 (SARS-COV-2) by means of dry powder inhaler through inhalation. The present invention also relates to methods for preparation of said pharmaceutical composition and the use of these active substances in symptoms caused by COVID-19, similar viral lung diseases.

There are several important definitions that should be considered while interpreting the claims and various aspects of the invention.

The inhaler device is preferably a single dose dry powder inhaler but may alternatively be a multi dose dry powder inhaler.

The term “powder” means a composition that consists of finely dispersed solid particles that are free flowing and capable of being readily dispersed in an inhalation device and subsequently inhaled by a subject so that the particles reach the lungs to permit penetration into the alveoli. Thus, the powder is said to be “respirable.” Preferably the average particle size is less than about 10 μm in diameter with a relatively uniform spheroidal shape distribution. More preferably the diameter is less than about 7.5 μm and most preferably less than about 5 μm. Usually the particle size distribution is between about 0.1 μm and about 5 μm in diameter, particularly about 2 μm to about 5 μm. In preferred embodiments, at least 50% (preferably at least 60%, more preferably at least 70%, still more preferably at least 80%, and most preferably at least 90%) of the total mass of active compounds which exits the inhaler device consists of particles within the desired diameter range.

The term “dry” means that the composition has a moisture content such that the particles are readily dispersable in an inhalation device to form an aerosol. This moisture content is generally below about 10% by weight (% w (w/w)) water, usually below about 5% w and preferably less than about 3% w.

The term “therapeutically effective amount” is the amount present in the composition that is needed to provide the desired level of therapeutic efficacy.

The term “physiologically effective amount” is that amount delivered to a subject to give the desired palliative or curative effect.

The term “pharmaceutically acceptable” carrier means that the carrier can be taken into the lungs with no significant adverse toxicological effects on the lungs.

In the context of the embodiments of the invention the term “active substance used against viral diseases” refers, without limitation, to an agent that kills a virus or that suppresses its ability to replicate and, hence, inhibits its capability to multiply and/or reproduce, or an agent that helps to reduce cytokine storm or harmful inflammatory responses. It can be interchangeably referred as anti-viral drug or anti-viral substance or anti-viral compound.

The individual compounds of the dry powder inhaler as described herein may be administered either sequentially or simultaneously. When administered simultaneously the individual compounds may be in separate compositions or a combined composition (i.e. admixed). Dry powder composition of the present invention contains a powder mix for inhalation active ingredient and a suitable powder base (carrier/diluent/excipient substance). The excipient can be selected from carriers, glidants, lubricants, additives, bulking agents, pH adjusters, buffers or protein stabilizers.

Conventional formulations for dry powder inhalation aerosols typically contain micronized drug of particle sizes small enough to enter the airways and be deposited in the lung. To make these highly cohesive and very fine particles dispersible, so called “carrier” particles are mixed with the drug particles. The carrier particles serve to increase the fluidization of the drug because the drug particles are normally too small to be influenced significantly by the airflow through the inhaler. The carrier particles thus improve the dose uniformity by acting as a diluent or bulking agent in the formulation. The purpose of the carrier is to prevent aggregation of the drug particles due to cohesive forces, primarily van der Waals forces arising from the instantaneous dipole moments between neighboring drug particles.

The pharmaceutically acceptable carrier for making dry powders can comprise any carriers or excipients useful for making dry powders and which are suitable for pulmonary delivery. The amount of the pharmaceutically acceptable carrier is that amount needed to provide the necessary stability, dispersibility, consistency and bulking characteristics to ensure a uniform pulmonary delivery of the composition to a subject in need thereof. Numerically the amount may be from about 70-100% w/w preferably 95.0% w/w to about 99.95% w/w, depending on the activity of the API.

Suitable carriers may be in the form of an amorphous powder, a crystalline powder, or a combination of amorphous and crystalline powders. At least one excipient as carrier is selected from carbohydrates, monosaccharides, fructose, galactose, glucose, D-mannose, sorbose, disaccharides, lactose, a-lactose monohydrate, trehalose, cellobiose, mono-, di- or poly-saccharides, sucrose, maltose, raffinose, melezitose, mannitol, lactitol, melibiose, cellobiose, dextrin, maltodextrin, dextran, sorbitol, galactitol, iditol, heptanol, fucoseitol, inositol, maltitol, lactitol, isomalt, maltotriose, maltotetraose, polyglycitol, xylitol, starch, citrates, amino acids, glycine, arginine, aspartic acid, glutamic acid, cysteine, lysine, L-leucine, isoleucine, trileucine, tartrates, methionine, organic salts, sodium citrate, sodium ascorbate, magnesium gluconate, sodium gluconate, tromethamine hydrochloride, zinc citrate, trisodium citrate, zinc chloride, polyvinylpyrrolidone, peptides, proteins, aspartame, human serum albumin, gelatin, alditols, phospholipids, diphosphotidylcholine, cyclodextrins, 2-hydroxypropyl-3-cyclodextrin, sugar ester and β-D-Mannitol (MA). The excipient can be any one or any combinations of said carriers.

At least one excipient as glidant or lubricant is selected from calcium stearate and magnesium stearate. The excipient can be any one or any combinations of said glidants or lubricants. Magnesium stearate, can be used to alter the surface properties of carrier particles and thereby improve the properties of dry powder formulations, to improve the moisture resistance of dry powders etc. If magnesium stearate is present in the composition, it is generally used in an amount of about 0.2 to 2%, e.g. 0.6 to 2% or 0.5 to 1.75%, e.g. 0.6%, 0.75%, 1%, 1.25% or 1.5% weight/weight (w/w), based on the total weight of the composition.

Additives, which are minor components of the composition of this invention, may be included for conformational stability during spray drying and for improving dispersibility of the powder. At least one excipient as additive is selected from hydrophobic amino acids, tryptophan, tyrosine, leucine, phenylalanine, fatty acid salts, sulfates, phosphates, polysorbates, polysorbate 80, polyoxyethylene, polyoxypropylene block copolymers, polyoxyethylene sorbitan monooleate, benzalkonium chloride, cetyltrimethylammonium bromide, sodium docusate, glyceryl monooleate, sorbitan esters, sodium laurel sulfate, polysorbate esters, phospholipids and bile salts. The excipient can be any one or any combinations of said additives. Some of the mentioned additives are used as surfactants in the present pharmaceutical composition. Preferred nonionic surfactants that are used as additives, can be polysorbates i.e polysorbate 80, and polyoxyethylene and polyoxypropylene block copolymers, are referred to as “poloxamers.” Polysorbates are described in the CTFA International Cosmetic Ingredient Dictionary as a mixture of sorbitol and sorbitan fatty acid esters condensed with ethylene oxide. Particularly preferred are a series of nonionic surfactants known as “Tweens”, in particular the surfactant known as “Tween 80”, a commercially available polyoxyethylene sorbitan monooleate. The presence of a surfactant (and preferably Tween 80) is necessary to reduce the electrostatic charge found in surfactant-free formulations, powder flow and to maintain a uniform solid state in the absence of initial crystallization.

At least one excipient as bulking agent is selected from polyvinylpyrrolidone K-25 (PVP) and polyvinyl alcohol 3-88 (PVA). The excipient can be any one or any combinations of said bulking agents.

At least one excipient as pH adjuster or buffer is selected from organic salts, sodium citrate and sodium ascorbate. The excipient can be any one or any combinations of said pH adjusters or buffers.

At least one excipient as protein stabilizer is selected from sucrose, trehalose and raffinose.

Active substance used against viral diseases is selected from favipiravir, umifenovir, molnupiravir, ritonavir, pimodivir, hydroxychloroquine, remdesivir, acyclovir, gancyclovir, valganciclovir, valacyclovir, famciclovir, penciclovir, vidarabine, cidofovir, ribavirin, adefovir, entecavir, brincidofovir, idoxuridine, trifluridine, brivudine, FV-100, sorivudine, lamivudine, lobucavir, telbivudine, clevudine, tenofovir disoproxil, tenofovir, alafenamide, zidovudine, peramivir, amantadine, rimantadine, interferon alpha, interferon beta, chloroquine, favilavir, lopinavir, triazavirin, efavirenz, atazanavir, saquinavir, dolutegravir, asunaprevir, simeprevir, grazoprevir, daclatasvir, etravirine, abacavir, danoprevir, telaprevir, darunavir, nelfinavir, indinavir, boceprevir, lomibuvir, raltegravir, amphotericin B, rifampin, rifabutin, isoniazid, prazinamide, ethambutol, albuvirtide, amprenavir, apricitabine, atovaquone, AT-527, ATR-002, azithromycin, balavir, baloxavir, bictegravir, BMS-955176, brilacidin, cabotegravir, cenicriviroc, censavudine, cobicistat, CS-8958, daclastavir, delaviridine, didanosine, docosanol, doravirine, edoxudine, elvitegravir, emtricitabine, enfuvirtide, famiclovir, fosamprenavir, foscarnet, fosfonet, fostemsavir, GS-9883, ibacitabine, laninamivir, ivermectin, letermovir, loviride, maraviroc, methisazone, moroxidine, N-(2-aminoethly)-1-aziridine ethane amine, N4-hydroxycytidine (EIDD-1931), nitazoxanide, piconivir, PRO 140, rilprivirine, rimantidine, sofosbuvir, stavudine, tenovir alafenamide, tenovir, alafenamide fumarate, tenovir disoproxil, tipranavir, tromantadine, TXA127, valaciclovir, VERU-111, viciviroc, viramidine, zalcatibine and salt forms thereof, and cyclodextrin complexes thereof and pharmaceutically acceptable derivatives thereof and water-soluble forms and suspensions thereof. The active substance used against viral diseases can be any one or any combinations of said active agents.

The active substance used against viral diseases can be 1-200 mg. For remdesivir 20-100 mg dose; for hydroxychloroquine 5-100 mg dose; for favipiravir 5-200 mg dose; for umifenovir, molnupiravir, ritonavir, or pimodivir 10-200 mg, more specifically 10-100 mg can be used.

In the present invention, excipients, complying with the requirements of Ph. Eur/USNF can be used. Excipients that are mentioned herein, can have different functionalities. One of the excipients, β-D-Mannitol (MA), is a hydrophilic excipient, characterized by high water-solubility, low toxicity, low hygroscopic profile and significant stability. Amother excipient, Polyvinylpyrrolidone K-25 (PVP), is a stabilizing agent; polyvinyl alcohol 3-88 (PVA) is a microfine coating material, L-leucine (LEU) is an amino acid that can be well co-spray dried with certain active compounds to modify the drug's aerolization behaviour.

Anhydrous glucose is already used in the marketed product Bronchodual (Boehringer). Sucrose, trehalose and raffinose are nonreducing sugars and as such have the advantage that they will not undergo the Maillard browning reaction with proteins. However, in contrast to lactose monohydrate, which was found to only take up moisture on its surface, a main difficulty with these sugars (especially with the more hygroscopic substances-sorbitol, maltitol and xylitol) has been attributed to their sensitivity to humidity.

To date, lactose has been employed as carrier in particular for crytalline actives and on some occusion for peptide/protein compounds. Lactose which is used for appropriate formulations is produced by crystallization, followed by grinding and screening to the required particle size.

In general, all carriers having the characteristics described in the pharmacopeia can be applied, but use of lactose is preferred. The lactose may be for example anhydrous lactose or a-lactose monohydrate. In one embodiment, the carrier is a-lactose monohydrate. The carrier when it is lactose e.g. a-lactose monohydrate, may form from about 91 to about 99% (91-99%), e.g. 97.7-99.0% or 91.0-99.2% by weight (weight/weight) of the composition. In general, the particle size of the carrier, for example lactose, will be much greater than the inhaled medicament within the present invention.

There are also some protein stabilizers used in the present pharmaceutical composition. Sucrose, trehalose and raffinose are non-reducing sugars and as such have the advantage that they will not undergo the Maillard browning reaction with proteins. Protection of protein structure is essential in both processing and storage of the final formulation. Sugars, such as trehalose and raffinose, have been employed to act as protein stabilizers. Sugars (particularly disaccharides), polyols, amino acids or organic salts have been used to protect native protein structure during drying processes, including spray-drying, and storage.

The pharmaceutical composition which is used for inhalation as dry powder is in the form of micronised powder. So, it is characterised by particles of few microns particle size. Poor flowability is also detrimental to the respirable fraction of the delivered dose being the active particles unable to leave the inhaler and remaining adhered to the interior of the inhaler or leaving the inhaler as large agglomerates, agglomerated particles, in turn, can not reach the bronchiolar and alveolar sites of the lungs. The uncertainty as to the extent of agglomeration of the particles between each actuation of the inhaler and also between inhalers and different batches of particles, leads to poor dose reproducibility as well.

The effectiveness of the medication depends to a large extent on the release of stable and high fine particle doses (FPD) from dry powder inhalants. FPD is the mass of a breathable dose with an aerodynamic particle size of less than 5 μm released from a dry powder inhaler, it is preferably % 20 in dry powder inhaler. Therefore, when inhaling one dose of any dry powder of a drug, it is important to obtain particles having a high aerodynamic particle size of fine particle percentage (FPF) with a mass of preferably less than 5 μm in the inhaled air. Most larger particles (>5 μm) do not enter many branches of the respiratory tract with air flow, but stick to the throat and upper respiratory tract, where the drug does not exhibit the expected effect and may be harmful to the user. It is also important to keep the dosage as accurate as possible to the user to maintain stable efficacy over time, and the dosage of the drug does not deteriorate during normal storage.

In the production of the pharmaceutical composition of the present invention that is suitable for inhalation with a dry powder inhaler device, API is suspended in a liquid with an excipient, followed by stirring, and the liquid is evaporated after the mixture is obtained. Mixing materials of different particle sizes is another method that teaches how to produce a uniform powder mixture with a special mixing method. Another method teaches continuous dosing into a mixer to obtain a uniform powder formulation. There are also methods that can be used to produce a homogeneous powder formulation of one or more excipients and a API, including using air or some other pharmaceutically acceptable gas as a suspension medium in a batch or continuous mixing step. Granularly mixed excipients are prepared with API and optionally one or more additional APIs. All these mentioned methods can be used in production of the pharmaceutical composition of the present invention.

The DPI of the present invention contains a predetermined amount of dry powder dose with a high FPD and enables selection of suitable high-quality excipients to obtain good water content and to form doses up to high FPD (e.g., using electric field splitting technology and conventional Volume packing method). Targeted particle size of the pharmaceutical composition is 1-5 μm.

The dry powder inhalers of the present invention may be, for example, reservoir dry powder inhalers, unit-dose dry powder inhalers, or pre-metered multi-dose dry powder inhalers. The compositions may be presented in unit dosage form for delivery by an appropriate dry powder inhaler. Dry powder compositions for topical delivery to the lung by inhalation may presented in capsules and cartridges of for example gelatine, or blisters of for example laminated aluminium foil. Packaging of the formulation may be suitable for unit dose or multi-dose delivery.

In one embodiment, a composition suitable for inhaled administration may be incorporated into a plurality of sealed dose containers provided on medicament pack(s) mounted inside an appropriate dry powder inhaler. The containers may be rupturable, peelable or otherwise openable one-at-a-time and the doses of the dry powder composition administered by inhalation on a mouthpiece of the inhalation device, as known in the art. The medicament pack may take a number of different forms, for instance a disk-shape or an elongate strip. A further delivery method for a dry powder inhalable composition is for metered doses of the composition to be provided in capsules (one dose per capsule) which are then loaded into an appropriate dry powder inhaler, typically by the patient on demand. The device has means to rupture, pierce or otherwise open the capsule so that the dose is able to be entrained into the patient's lung when they inhale at the device mouthpiece.

### REFERENCES

1. Bocan T M, Basuli F, Stafford R G, Brown J L, Zhang X, Duplantier A J, Swenson R E. Synthesis of [¹⁸F] Favipiravir and Biodistribution in C3H/HeN Mice as Assessed by Positron Emission Tomography. Sci Rep. 2019 Feb. 11;9(1):1785. doi: 10.1038/s41598-018-37866-z. PMID: 30741966; PMCID: PMC6370782.

1. A pharmaceutical composition in inhalable pre-metered dry powder form comprising
at least one excipient,
at least one active substance used against viral diseases selected from favipiravir, umifenovir, molnupiravir, ritonavir, pimodivir, hydroxychloroquine, remdesivir, acyclovir, gancyclovir, valganciclovir, valacyclovir, famciclovir, penciclovir, vidarabine, cidofovir, ribavirin, adefovir, entecavir, brincidofovir, idoxuridine, trifluridine, brivudine, FV-100, sorivudine, lamivudine, lobucavir, telbivudine, clevudine, tenofovir disoproxil, tenofovir, alafenamide, zidovudine, peramivir, amantadine, rimantadine, interferon alpha, interferon beta, chloroquine, favilavir, lopinavir, triazavirin, efavirenz, atazanavir, saquinavir, dolutegravir, asunaprevir, simeprevir, grazoprevir, daclatasvir, etravirine, abacavir, danoprevir, telaprevir, darunavir, nelfinavir, indinavir, boceprevir, lomibuvir, raltegravir, amphotericin B, rifampin, rifabutin, isoniazid, prazinamide, ethambutol, albuvirtide, amprenavir, apricitabine, atovaquone, AT-527, ATR-002, azithromycin, balavir, baloxavir, bictegravir, BMS-955176, brilacidin, cabotegravir, cenicriviroc, censavudine, cobicistat, CS-8958, daclastavir, delaviridine, didanosine, docosanol, doravirine, edoxudine, elvitegravir, emtricitabine, enfuvirtide, famiclovir, fosamprenavir, foscarnet, fosfonet, fostemsavir, GS-9883, ibacitabine, laninamivir, ivermectin, letermovir, loviride, maraviroc, methisazone, moroxidine, N-(2-aminoethly)-1-aziridine ethane amine, N4-hydroxycytidine, nitazoxanide, piconivir, PRO 140, rilprivirine, rimantidine, sofosbuvir, stavudine, tenovir alafenamide, tenovir, alafenamide fumarate, tenovir disoproxil, tipranavir, tromantadine, TXA127, valaciclovir, VERU-111, viciviroc, viramidine, zalcatibine and salt forms thereof, and cyclodextrin complexes thereof and pharmaceutically acceptable derivatives thereof and water-soluble forms and suspensions thereof
to be locally administered to the lungs for use in the treatment of symptoms of viral lung diseases including COVID-19 caused by Severe Acute Respiratory Syndrome-Coronavirus-2 (SARS-COV-2) by means of dry powder inhaler through inhalation.

2. The pharmaceutical composition according to claim 1, wherein it comprises 1-200 mg of active substance used against viral diseases.

3. The pharmaceutical composition according to claim 1, wherein at least one excipient is selected from carriers, glidants, lubricants, additives, bulking agents, pH adjusters, buffers or protein stabilizers.

4. The pharmaceutical composition according to claim 3, wherein said carriers are carbohydrates, monosaccharides, fructose, galactose, glucose, D-mannose, sorbose, disaccharides, lactose, a-lactose monohydrate, trehalose, cellobiose, mono-, di- or poly-saccharides, sucrose, maltose, raffinose, trehalose, melezitose, mannitol, lactitol, melibiose, cellobiose, dextrin, maltodextrin, dextran, sorbitol, galactitol, iditol, heptanol, fucoseitol, inositol, maltitol, lactitol, isomalt, maltotriose, maltotetraose, polyglycitol, xylitol, starch, citrates, amino acids, glycine, arginine, aspartic acid, glutamic acid, cysteine, lysine, L-leucine, isoleucine, trileucine, tartrates, methionine, organic salts, sodium citrate, sodium ascorbate, magnesium gluconate, sodium gluconate, tromethamine hydrochloride, zinc citrate, trisodium citrate, zinc chloride, polyvinylpyrrolidone, peptides, proteins, aspartame, human serum albumin, gelatin, alditols, phospholipids, diphosphotidylcholine, cyclodextrins, 2-hydroxypropyl-3-cyclodextrin, sugar ester and B-D-Mannitol (MA).

5. The pharmaceutical composition according to claim 3, wherein said glidants or lubricants are calcium stearate and magnesium stearate.

6. The pharmaceutical composition according to claim 3, wherein said additives are hydrophobic amino acids, tryptophan, tyrosine, leucine, phenylalanine, fatty acid salts, sulfates, phosphates, polysorbates, polysorbate 80, polyoxyethylene, polyoxypropylene block copolymers, polyoxyethylene sorbitan monooleate, benzalkonium chloride, cetyltrimethylammonium bromide, sodium docusate, glyceryl monooleate, sorbitan esters, sodium laurel sulfate, polysorbate esters, phospholipids and bile salts.

7. The pharmaceutical composition according to claim 3, wherein said bulking agents are polyvinylpyrrolidone K-25 (PVP) and polyvinyl alcohol 3-88 (PVA).

8. The pharmaceutical composition according to claim 3, wherein said pH adjusters or buffers are organic salts, sodium citrate and sodium ascorbate.

9. The pharmaceutical composition according to claim 3, wherein said protein stabilizers are sucrose, trehalose and raffinose.

10. The pharmaceutical composition according to claim 4, wherein the percentage of the carrier is between 70-100% by weight with respect to the total weight (w/w) of the composition.

11. The pharmaceutical composition according to claim 10, wherein the percentage of the carrier is between 95.0-99.95% w/w.

12. The pharmaceutical composition according to claim 10, wherein said carrier is lactose and percentage of the carrier is between 91-99% w/w.

13. The pharmaceutical composition according to claim 4, characterized in that the carrier is in amorphous powder form or a crystalline powder form or a combination of amorphous and crystalline powder form.

14. The pharmaceutical composition according to claim 1, wherein the composition has a particle size of 0.1-10 μm.

15. The pharmaceutical composition according to claim 14, wherein the composition has a particle size of 7.5 μm.

16. The pharmaceutical composition according to claim 14, wherein the composition has a particle size of 0.1-5 μm.

17. The pharmaceutical composition according to claim 14, wherein the composition has a particle size of 2-5 μm.

18. The pharmaceutical composition according to claim 1, wherein the composition has a moisture content below 10% by weight (w/w).

19. The pharmaceutical composition according to claim 18, wherein the composition has a moisture content below 5% w/w.

20. The pharmaceutical composition according to claim 1, wherein the composition has a moisture content below 3% w/w.

21. The pharmaceutical composition according to claim 5, said glidant or lubricant is magnesium stearate and the amount of magnesium stearate is between 0.2 to 2% w/w based on the total weight of the composition.

22. The pharmaceutical composition according to claim 21, said glidant or lubricant is magnesium stearate and the amount of magnesium stearate is between 0.6 to 2% w/w based on the total weight of the composition.

23. The pharmaceutical composition according to claim 21, said glidant or lubricant is magnesium stearate and the amount of magnesium stearate is between 0.5 to 1.75% w/w based on the total weight of the composition.
